(19) United States
(12) Patent Application Publication
(10) Pub. No.: US 2024/0004876 A1
(43) Pub. Date: Jan. 4, 2024

(54) METHOD AND SYSTEM FOR ASSISTING WITH THE SPECIFICATION OF A CONTEXT-ADAPTIVE BEHAVIOR OF A SYSTEM BY AN END USER

(71) Applicant: HOCHSCHULE DÜSSELDORF, Düsseldorf (DE)

(72) Inventor: Manfred Wojciechowski, Viersen (DE)

(21) Appl. No.: 18/252,320

(22) PCT Filed: Oct. 29, 2021
(86) PCT No.: PCT/EP2021/080083

§ 371 Date: May 9, 2023

(30) Foreign Application Priority Data
Nov. 10, 2020 (DE) 102020129640.1

(51) Int. Cl. G06F 16/242; G06F 16/2455

(52) U.S. Cl. CPC G06F 16/2448; G06F 16/24553

(57) ABSTRACT

A method for assisting with the specification of a context-adaptive behavior of a system by an end user using an input unit includes: provision of an input unit, a sensor system and a computing unit; capture of at least one item of context information in the surroundings of the end user by means of a sensor system; creation of a context subspace; refinement of a predefined situation description by the end user; termination of the refinement by the end user; combination of private and public components of the definition of the context subspace, together with the extension resulting from the refinement by the end user, into a query in the query language; compilation of the query by the computing unit using a context server; and execution of the query by means of the computing unit.

The invention relates to a method for assisting with the specification of a context-adaptive behavior of a system by an end user. The invention also relates to a system that is designed to execute the method. The invention furthermore relates to a computer program product, comprising commands which, when the program is executed by the system, cause said system to execute the method.

The invention lies in the field of research into context adaptivity. Initial research work in this field took place as early as in the 1990s. The term was first used in 1994 by Schilit and Theimer (Schilit, B. N.: Theimer, M. M.: “Disseminating active map information to mobile hosts.” in: Network, IEEE8 (1994), September/October, No. 5, pp. 22-32). Context adaptivity is to be considered in conjunction with systems that are capable of independently adapting themselves to states of, and changes in, their environment. The definition of context adaptivity that is nowadays common in scientific research originated from Dey and Abowd in 1999: “A system is context-aware if it uses context to provide relevant information and/or services to the user, where relevancy depends on the user's task” (Dey, Anind K.; Abowd, Gregory D.: Towards a Better: Understanding of Context and Context-Awareness/Georgia Institute of Technology, Atlanta, GA, USA 30332 0280). They furthermore define context as follows: “Context is any information that can be used to characterize the situation of an entity. An entity is a person, place, or object that is considered relevant to the interaction between a user and an application, including the user and applications themselves.” A context-adaptive system will thus utilize items of information relating to entities or their relationships to one another in order, from these, to draw inferences regarding situations in which the system can automatically offer relevant items of information or functions to the user. Such items of information may be gathered using sensor systems, for example. An example of a context-adaptive system from the “Smart Home” sector is an emergency call system which, on the basis of sensor systems, identifies defined emergency situations, for example a fall of the resident, and correspondingly places an emergency call. In the field of research into context adaptivity, it is possible to identify different activities. One sub-field is concerned with developing and using sensor systems to derive items of context information. Here, research is for example being carried out into methods for using different sensors to more reliably derive items of context information, in the sense of sensor fusion. Methods for identifying situations on the basis of classification methods also fall under this category. A further sub-field is concerned with architectures and platforms for developing context-adaptive systems. Different approaches for this have already been developed. For example, various context servers have been developed and published, but these are used only for research purposes. An important question here is how context can be modeled in order to be able to represent entities and their relationships to one another, and their characteristics, in such a platform. Here, too, there are various approaches that range from simple key value models to semantic models in the form of ontologies.

Context metamodels that are concerned with the ability of the end user to define the context-adaptive behavior of a system are known from the prior art, for example from the dissertation by Manfred Wojciechowski: “Kontextmodellierung für das Ambient Assisted Living” [“Context modeling for ambient assisted living” ], TU Dortmund, 2011. This is referred to as “end user programming”. The background to these activities is that studies have shown that complete automation using context-adaptive systems, without any control by the end user, is not in the end user's interests, and will therefore not be accepted.

The article “Exploring end user programming needs in home automation” by Bricht et al., from ACM Transactions on Computer-Human Interaction (TOCHI) 24.2 (2017): 1-35, gives an overview of the demands on home automation with regard to “end user programming”. The authors furthermore describe the two currently predominantly pursued approaches of “rule-based programming” and “process-oriented programming”. In both approaches, the end user can use a graphical notation to describe the desired context-adaptive behavior, either in the form of IF-THEN rules with logical operators, or in the form of chronological sequences by way of a control graphic. The article describes a study with 18 test subjects with regard to the usability of these approaches for the end user. The study indicates that the rule-based approach appears adequate for the end user to define very simple automation tasks, because it is perceived to be well structured. This approach is however perceived to be too restricted in the case of more complex automation tasks. Furthermore, half of the participants in the study experienced problems with the logic-based linking of conditions. There are various examples for the approach of “rule-based programming”, for example from Huang and Cakmak (Huang, Justin and Maya Cakmak, “Supporting mental model accuracy in trigger-action programming”, Proceedings of the 2015 ACM International Joint Conference on Pervasive and Ubiquitous Computing, 2105) or from Zhang and Brugge (Zhang, Tao, and Bernd Brugge, “Empowering the user to build smart home applications”, ICOST 2004 International Conference on Smart Home and Health Telematics, 2004). The “process-oriented” approach was evaluated by half of the participants as being easy and straightforward. Two-thirds of the participants found the notation of the process orientation to be intuitive and easy to understand. Only one participant found the automation tasks defined using a control graphic to be structured. Two of 9 participants were unsure when to use AND or OR links in the process description. For the implementation of both approaches, it was necessary for the end users to be capable of understanding the rule-based approach and the process-oriented approach together with graphical notation, and to express the desired automation tasks, taking into consideration the equipment present, in said graphical notation. The studies showed that there is still a demand for research, and no current approach offers a satisfactory solution to the problem.

Outside the research sector, there are various products, and also free software, that can be used to implement home automation. A first category is formed by so-called Smart Home Hubs. These are software platforms that allow the integration of different communication protocols, sensors and actuators, and the implementation of context-adaptive systems on this basis. One example of this is openHAB. This allows rule-based creation of home automation tasks by the end user using a web interface. This does not support any graphical notation, and involves the same problems as described in the study by Brich et al. Further examples of such hubs are the Samsung SmartThings Hub or the Insteon Hub.

A further category is formed by speech assistants from Amazon or Google. These make it possible to use speech control to activate different profiles and thus, for example via additional adapters, to control appliances in the home, for example using the Amazon Echo. Context-adaptive home automation can be implemented here only to a limited extent, for example in the form of time-controlled activities. In August 2018, a Contact and Motion Sensor API for Amazon Alexa was announced, which allows the connection of sensors for the initiation of defined automation scenarios. Simple context-adaptive automation can also be implemented in this way.

Both for certain Smart Home Hubs, such as openHAB, but also for the speech assistants from Amazon or Google, it is possible to create and carry out simple rule-based automation tasks using the service provider IFTTT for individually linking web applications. Technically experienced users can create such automation tasks by way of rule-based IF-THEN commands in the form of formulae, and then also make these available to other users.

As already described in the prior art, both rule-based and process-oriented approaches to “end user programming” are not yet satisfactory, because they require technical knowledge and an understanding of the semantics of the graphical notation. The rule-based approach can be suitable for simple automation tasks. However, if it is sought to define more complex automation tasks, which for example necessitate a combination of different sensors, there is not yet a suitable approach by which the end user can do so.

Previous attempts to reach a solution have been described in the prior art. Furthermore, in 2004, Dey et al. (Dey, Anind K., et al., “a CAPpella: programming by demonstration of context-aware applications”, Proceedings of the SIGCHI conference on human factors in computing systems, 2004) described an approach in which an AI was used that enabled the end user to demonstrate, in the surroundings, what situations are relevant for the triggering of events. For this purpose, the end user was able to record the situation using a camera, microphone and other sensors, subsequently select the crucial features, for example image segments, and then teach the situation to the AI.

The document “Kontextmodellierung fir das ambient assisted living” [“Context modeling for ambient assisted living” ] by Manfred Wojciechowski from May 2011 describes an approach which takes a situation description predefined by experts as a starting point, which the end user can navigate in order to select the desired situation. FIG. 1 shows, by way of example, an image of navigation through a situation description from the aforementioned prior art.

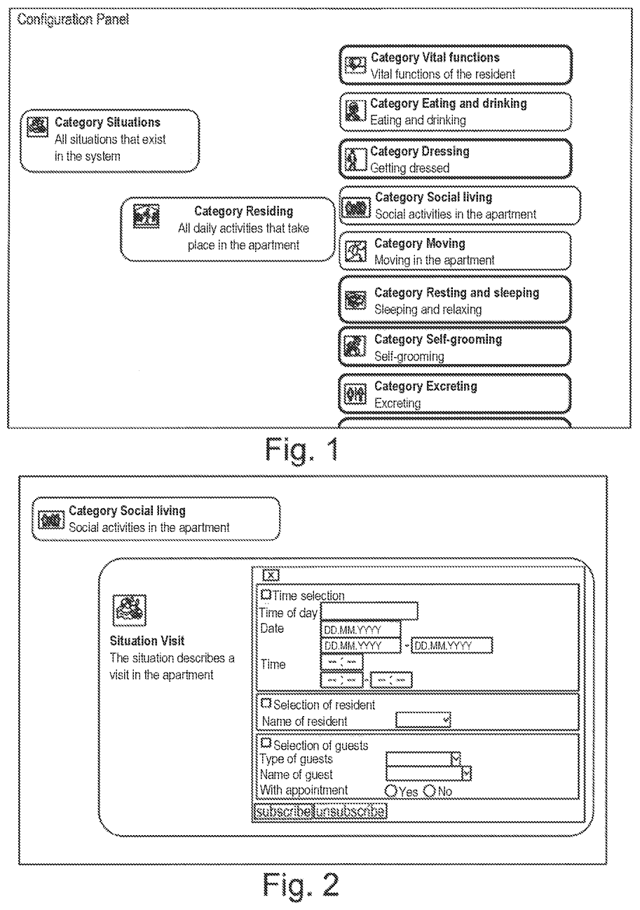

After selecting a situation description, the user can adapt this to themselves by refinement within the specified scope, for example by indicating a cardinality (max. 2 persons), selecting a specialized context entity (“carer” as a specialization of a “person”) or applying limitations to attributes (time: after 6 o'clock). FIG. 2 shows a corresponding image of adaptation of a situation description from the prior art.

This form of “end user programming” restricts the user to the situations predefined by experts.

The object on which the invention is based consists in further refining predefined situation descriptions through the inputting of items of information using an input unit, in order, from these, to make it possible for situation descriptions defined by the end user to be executed by a context server using a computing unit.

Therefore, according to the invention, a method for assisting with the specification of a context-adaptive behavior of a system by an end user using an input unit is specified, the method comprising the following steps:
provision of an input unit for the inputting of items of information by the end user,
provision of a sensor system, the sensor system being configured to capture at least one item of context information in the surroundings of the end user,
provision of a computing unit, the computing unit being configured to receive the items of information provided at the input unit and to receive the items of context information captured by means of the sensor system, the computing unit furthermore being configured to create a situation description from the items of context information captured by means of the sensor system and to create a context subspace,
capture of at least one item of context information in the surroundings of the end user by means of the sensor system,
creation of a context subspace by means of the computing unit, the context subspace specifying all possible states of context entities and their context relationships to one another, the state of a context entity being described by at least one context attribute, the possible values of the context attribute being specified by a data type, the context relationships being described by at least one context attribute, the possible values of the context attribute being specified by a data type, context attributes of context entities and context attributes of context relationships being described by means of the items of context information captured by the sensor system, the context subspace describing the greatest possible extract from a context model that can describe a situation under all possible circumstances,
   the context subspace being defined by means of a query language that directly represents an underlying metamodel,
   the query language providing a query for context entities of a particular type and features for inspection, taking into consideration the inheritance hierarchies defined in the metamodel, and the query language furthermore providing a query for relationships between context entities and features for inspection,
   a definition of the context subspace by means of the query language being composed of at least one public component and one private component,
   the private component defining the underlying connections between the context entities and their relationships, which make up a context subspace, and
   the public component marking parts the definition which can be made known to the end user, and describing a predefined situation description,
   it being possible to retroactively extend the public component of the definition of the context subspace in the query language such that refinements can be added to the definition by the end user,
refinement of the predefined situation description by the end user by inputting of items of information using the input unit,
termination of the refinement by the end user,
combination of the private components and the public components of the definition of the context subspace, and of the extension resulting from the refinement by the end user, into a query in the query language,
compilation of the query by the computing unit using a context server,
execution of the query by means of the computing unit.

It is the intention for the end user to be rendered capable, even without programming knowledge, of easily expressing complex context conditions for automation tasks, which can be monitored by a corresponding context server and used for controlling these automation tasks. According to the invention, provision is therefore made for a new context server to be implemented, the method executed by a computer making it possible for a situation description to be executed by a context server, such that the situation described by the end user can be monitored by the server.

Here, a sensor system is an essential constituent part, for example of the infrastructure of an intelligent environment. Sensors provide items of context information that are required for context adaptivity. Numerous sensors already exist for capturing different items of information.

The context model is a basis for the formal capture of items of context information. Metamodels are the implicit or explicit basis of these context models. Common observable characteristics of context entities can be expressed in the context model. These may be formulated in the form of an inheritance hierarchy. To describe them, the metamodel may use the definition of generalization or specialization relationships between context entities.

A context entity defines the object regarding which an observation is made or an item of context information is given. Said context entity is thus the focal point of the observation. Said context entities may in particular be persons, locations and items that are relevant for the context adaptivity of systems. It is thus possible for potentially any type of object from the real world to be recorded as an entity in a context model.

A context relationship explicitly describes a relationship between one or more context entities at the model level. Relationships on the metamodel level, for example the generalization/specialization relationship between entities, are not expressed by this model element. The context relationship model element does not yet make available the information relating to a specific relationship, but focuses the observation on this. For example, if a context relationship between a person and a space is defined, this does not mean that the person is positioned in the space. Rather, it expresses that such a relationship between the person and a space can be observed.

At the model level, a context attribute defines the item of information which can be observed and which is relevant for describing a context entity or context relationship. The value in a context attribute describes the state of an entity or relationship in the defined aspect.

According to the invention, a system is also specified, the system comprising: an input unit that is configured for the inputting of items of information by the end user, a sensor system that is configured to capture at least one item of context information in the surroundings of the end user, a computing unit that is configured to receive the items of information provided at the input unit and to receive the items of context information captured by means of the sensor system, the system being designed to execute the method described above.

In one preferred embodiment of the invention, the sensor system comprises at least one sensor, the sensor being selected from the group comprising: optical sensors, audio sensors, motion sensors, acceleration sensors, locating sensors, magnetic field sensors, orientation sensors, proximity sensors, touch sensors, sensors for temperature, humidity and air pressure, weight sensors, sensors for identifying an activity, gas sensors, odor sensors, biosensors, in particular sensors for capturing vital parameters.

Using simple optical sensors, items of information relating to the intensity, darkness, reflection and color temperature of the ambient light can be collected. Special types may measure the wavelength or segments in the light spectrum. From the pattern of the change in light, indications regarding the surroundings can be obtained, for example the 50 Hz flickering of a television. If multiple light sensors with different spatial orientation are combined at one sensor node, the light distribution can be used as a starting point for deriving further items of context information, such as movement. If more complex sensors are used, for example a CMOS camera in conjunction with a signal processor, then it is possible, depending on the complexity of the algorithms used, to derive further items of context information relating to the surroundings, for example movement, color distribution, identification of objects, landscapes and persons.

With the aid of microphones, items of acoustic information, including sound intensity and frequency, can be gathered from the surroundings. Different acoustic types, for example noise, music and speech, can be registered. Using complex algorithms, it is possible to draw inferences regarding the situation in the surroundings, for example traffic noises and cries of children. The type of music can also be identified. Speech recognition can likewise be used as a basis for deriving items of context information. With the aid of multiple microphones, it is also possible to localize the acoustic source.

Artifacts can be equipped with sensors for measuring movement and acceleration. These may for example include articles of clothing, walking aids or mobile telephones. In this way, items of context information relating to the user of these artifacts, such as sporty activity or a fall, can be derived.

Numerous sensors exist for locating persons and objects. Aside from location technologies that are locally present in the home environment, for example using radio frequency identification (RFID) or infrared location, consideration should also be given to location services offered by external service providers, such as mobile service providers. Location is a commonly used item of context information.

Sensors that react to the Earth's magnetic field can be used for directional determination, which then determines the orientation of a device or the direction of a movement.

Numerous sensors exist which can be used to measure the distance between objects or persons and a reference point. This may be performed using a laser, for example. By means of a pressure-sensitive surface, it is possible to determine when an object is touched by the resident. This may also be achieved through the use of a voltage-sensitive sensor that can also determine skin conductivity and muscle tension.

A wide range of inexpensive sensors exist for measuring the temperature of the surroundings, of an object or of a person. Humidity and air pressure in an environment can likewise be ascertained using inexpensive sensors. Aside from actively determining these items of environmental information using sensors, weather services available on the Internet may also be used as information sources for these items of context information.

The weight of an object or of a person can be measured using simple sensor systems. Aside from the absolute weight, the variance of the measured values can provide interesting items of context information. For example, with a spatially distributed arrangement of weight sensors, the movement of persons can be tracked.

Identifying general activity within a space is possible using simple sensor systems. This is performed for example using “passive infrared sensors” (PIR) which check whether persons are moving within the range.

Furthermore, numerous specialized sensors exist that can be used to detect particular gases, alcohol or food items. To identify odors, specialized devices exist which combine numerous sensors with one another. These are presently used primarily in the food industry.

Biosensors can be used to determine vital parameters of persons, which include for example blood pressure, pulse or blood sugar level. Many of these can already be purchased for home use.

In a further preferred embodiment of the invention, the input unit comprises at least one touch-sensitive screen and/or one touch-sensitive panel. Using a touch-sensitive screen and/or a touch-sensitive panel, the end user can easily refine or modify a predefined situation description, for example.

According to the invention, a computer program product is also specified, comprising commands which, when the program is executed by the above-described system, cause said system to execute the above-described method.

The invention will be discussed in more detail below on the basis of preferred exemplary embodiments, with reference to the appended drawing.

In the drawing:

FIG. 1 shows a screenshot of navigation through a situation description from the prior art,

FIG. 2 shows a screenshot of adaptation of a situation description from the prior art,

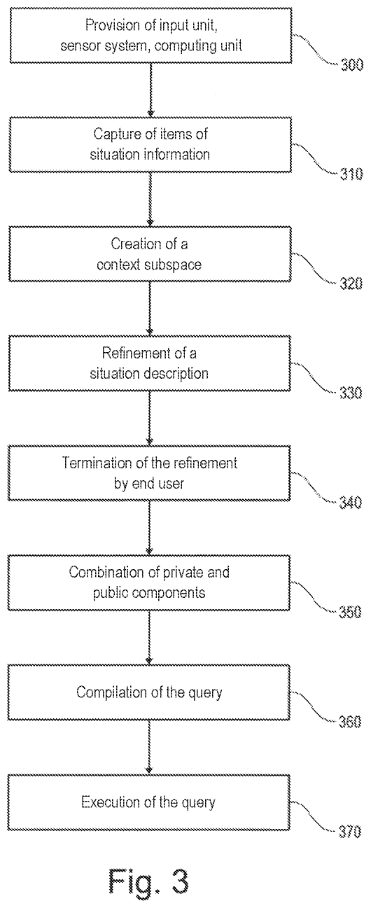

FIG. 3 shows a flow diagram of the method according to an exemplary embodiment of the invention,

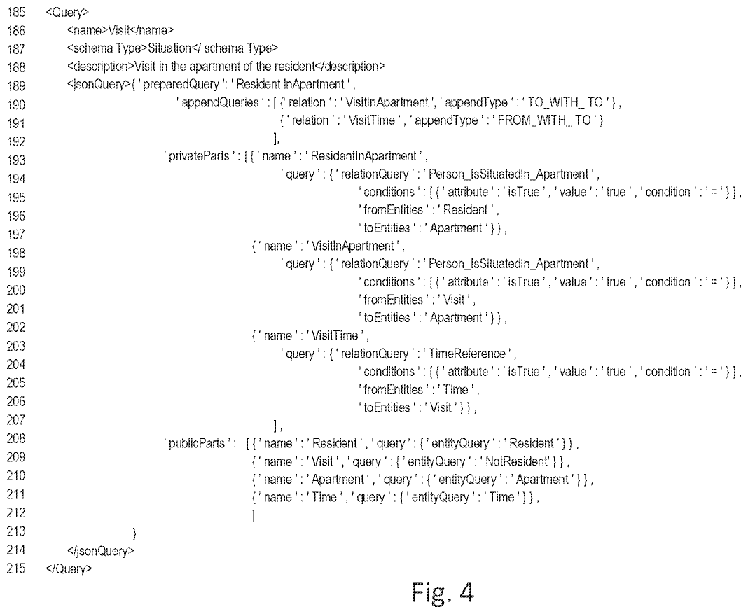

FIG. 4 shows an extract from the source code of a definition of a situation “Visit” according to an exemplary embodiment of the invention,

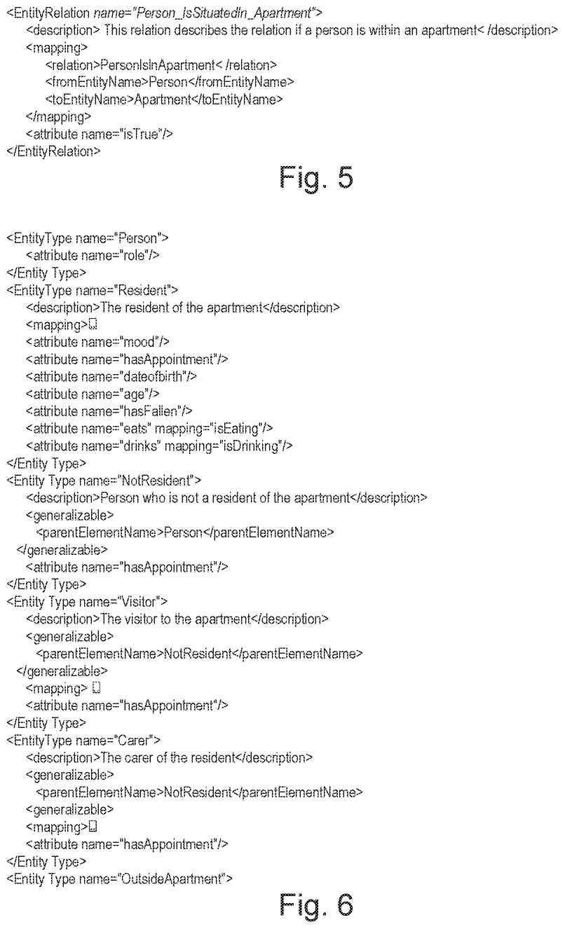

FIG. 5 shows an extract from the source code of a definition of a situation by a metamodel according to a further exemplary embodiment of the invention,

FIG. 6 shows an extract from the source code of a definition of the context identity by the metamodel according to a further exemplary embodiment of the invention,

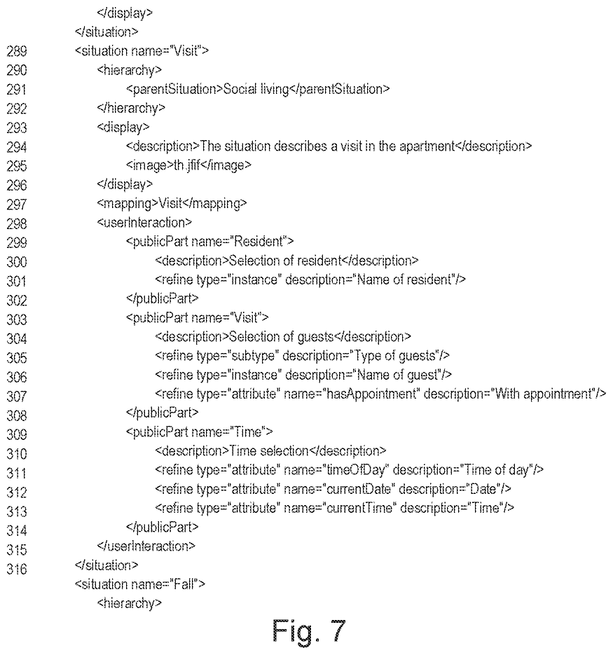

FIG. 7 shows an extract from the source code of a definition of a situation description and refinement options according to a further exemplary embodiment of the invention,

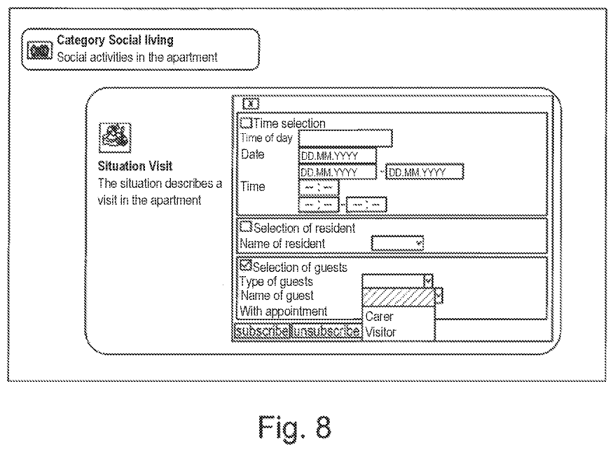

FIG. 8 shows a screenshot of a graphical interface with a selection of a specialization according to an exemplary embodiment of the invention.

FIG. 3 shows a flow diagram of a method according to an exemplary embodiment of the invention. The method for assisting with the specification of a context-adaptive behavior of a system by an end user starts, in step 300, with the provision of an input unit for the inputting of items of information by the end user. Also, in this step, a sensor system is provided, the sensor system being configured to capture at least one item of context information in the surroundings of the end user. Furthermore, a computing unit is provided, the computing unit being configured to receive the items of information provided at the input unit and to receive the items of context information captured by means of the sensor system, the computing unit furthermore being configured to create a situation description from the items of context information captured by means of the sensor system and to create a context subspace.

In step 310, at least one item of context information in the surroundings of the end user is captured by means of the sensor system. Here, the sensor system comprises at least one sensor, the sensor being selected from the group comprising: optical sensors, audio sensors, motion sensors, acceleration sensors, locating sensors, magnetic field sensors, orientation sensors, proximity sensors, touch sensors, sensors for temperature, humidity and air pressure, weight sensors, sensors for identifying an activity, gas sensors, odor sensors, biosensors, in particular sensors for capturing vital parameters. By using different sensors, items of context information in the surroundings of the end user can be exactly captured.

In step 320, a context subspace is firstly defined for the method, which context subspace describes an extract from a context space that defines the combination of all possible states of context entities and their relationships to one another. The definition of the context subspace is extended such that said context subspace now describes the greatest possible extract from the context model that can describe a situation under all possible circumstances. For example, the time aspect is not imperatively necessary for characterizing a visit, but, for the end user, to define the individual requirement, it may be necessary to also take the time of the visit into consideration as a basis for home automation, for example. Such a context subspace is defined in step 310 by means of a query language which directly represents the underlying metamodel and can therefore be executed by the context server. A description of a situation created by means of this query language can be executed in the context server in order to check whether the situation thereby described has been identified. This query language may offer the following possibilities:

A query for context entities of a particular type and features for inspection, taking into consideration the inheritance hierarchy defined in the metamodel, and a query for relationships between context entities and features for inspection. The definition of the context subspace by means of the query language may be composed of public and private components. Private components define the underlying connections between the context entities and their relationships, which make up a context subspace. These conceal the connections of the context model from the end user. The public components mark parts of the definition which can be made known to the user and which can be further refined by the user at a user interface. It must be possible for the public components of the definition of the context subspace to be retroactively extended in the query language, such that, for example, the end user's refinements can be added to the definition.

In step 330, the predefined situation description is refined by the end user by inputting of items of information using the input unit. In step 340, the inputting of the refinement by the end user is terminated.

In the subsequent step 350, the private and the public components of the definition, including the extension resulting from the refinement by the end user, are combined into a query in the query language, and in step 360 these are subsequently compiled by the computing unit using a context server. The query is subsequently executed by means of the computing unit in step 370.

The states of a context entity, for example of a person, may be described by attributes, the possible values of which are specified by a data type, for example their pulse. Context entities may have a relationship to one another, and the state of such a relationship may likewise be described by attributes, for example a person is situated in a space at a point in time. A situation is now described by an extract from the context space that can be characterized all characteristics, which are relevant for this situation, between the context entities relevant in said situation and their relationships to one another. For example, a situation “Visit” could be defined as follows:
Resident is in the apartment
A person who is not the resident is also in the apartment

The context entities “Person who is a resident”, “Person who is not a resident” and “Apartment” can be made known to the context server via the metamodel. Specific context entities such as “Max Müller” is a resident can likewise be made known in the context server. The context relationship “Person is in apartment” can be made known to the context server via the metamodel. The specific states of this relationship may be signaled in the context server by means of a sensor system.

The definition of the context subspace is extended such that said context subspace describes the greatest possible extract from the context model that can describe a situation under all possible circumstances. For example, the time aspect is not imperatively necessary for characterizing a visit, but, for the end user, to define the individual requirement, it may be necessary to also take the time of the visit into consideration as a basis for home automation.

Such a context subspace is defined by means of a query language which directly represents the underlying metamodel and can therefore be executed by the context server. A description of a situation created by means of this query language can be executed in the context server in order to check whether the situation thereby described has been identified. This query language may offer at least the following possibilities:
The query for context entities of a particular type and features for inspection, taking into consideration the inheritance hierarchy defined in the metamodel.
The query for relationships between context entities and features for inspection.

FIG. 4 illustrates the definition of a context subspace as an exemplary embodiment of the invention for the situation “Visit” in the query language of the context server. A context subspace is described here by a “PreparedQuery”. Aside from its main part, this also includes the private components “privateParts” and the public components “publicParts”. The main part of the query is composed of a combination of the queries “ResidentInApartment”, “VisitinApartment” and “VisitTime” (lines 188-191). These are defined in the private components (lines 192-207). All three queries check relationships between context entities. In the query language, these are named “relationQuery”. The relationships “Person_IsSituatedIn_Apartment and “TimeReference” are known in the context server via the metamodel, and are completed here by way of a sensor system.

These queries for relationships between context entities build upon queries from context entities that are defined in the public components, as shown on lines 208-212 in FIG. 4. In the query language, these are named “entityQuery”. The context entities “Resident”, “NotResident”, “Apartment” and “Time” are known in the context server via the metamodel, and are completed here by way of a sensor system.

FIG. 5 shows an extract from a source code of a definition of a situation by a metamodel according to a further exemplary embodiment of the invention. Here, FIG. 5 shows a simplified example of the definition of the corresponding context entities via the metamodel, such as may be implemented in the context server in accordance with an exemplary embodiment of the invention.

The context subspace thus defined can be evaluated by the context server. For the refinement by the end user, the metamodel provides here for the situation description and the refinement options to be described on the “user interface layer”. An example of this description, such as is implemented in the context server, is given in FIG. 6. A reference to the context subspace that is used is given in this description on line 297. This name corresponds to the name of the definition on line 186 in FIG. 4. The options for refinement by the end user are defined on lines 298-315. The names of the public parts on lines 299, 303 and 309 correspond to those of the definition on lines 208, 209 and 211 in FIG. 4. The refinement options are furthermore stated in the public parts, for example “instance”, “subtype” and “attribute”. This description is conveyed via a user interface. If, for example, the user selects the type of guests as on line 305 in FIG. 7, then the available specializations of the context entity are requested from the metamodel by the context server and are displayed to the user. FIG. 8 shows an image of a graphical interface with a selection of a specialization according to an exemplary embodiment of the invention.

If the user selects “Carer” as the specific type, then the public part “Visit” of the definition on line 209 in FIG. 4 is extended as follows by the context server via the user interface:
{“entity Query”: “NotResident”, “subtype”: “Carer”}

After the refinement by the end user has been terminated, the private and the public components of the definition, including the extension resulting from the refinement by the end user, are combined, are compiled by the context server using the computing unit, and are executed by means of the computing unit.

The various embodiments described above serve merely for illustrative purposes and are not intended to limit the invention. Experts will readily recognize various modifications and alterations that may be made to the present invention without following the exemplary embodiments and systems illustrated and described here and without departing from the scope of the present disclosure.

1. A method for assisting with the specification of a context-adaptive behavior of a system by an end user, the method comprising the following steps:
provision of an input unit for the inputting of items of information by the end user,
provision of a sensor system, the sensor system being configured to capture at least one item of context information in the surroundings of the end user,
provision of a computing unit, the computing unit being configured to receive the items of information provided at the input unit and to receive the items of context information captured by means of the sensor system, the computing unit furthermore being configured to create a situation description from the items of context information captured by means of the sensor system and to create a context subspace,
capture of at least one item of context information in the surroundings of the end user by means of the sensor system,
creation of a context subspace by means of the computing unit, the context subspace specifying all possible states of context entities and their context relationships to one another, the state of a context entity being described by at least one context attribute, the possible values of the context attribute being specified by a data type, the context relationships being described by at least one context attribute, the possible values of the context attribute being specified by a data type,
context attributes of context entities and context attributes of context relationships being described by means of the items of context information captured by the sensor system,
the context subspace describing the greatest possible extract from a context model that can describe a situation under all possible circumstances,
the context subspace being defined by means of a query language that directly represents an underlying metamodel,
the query language providing a query for context entities of a particular type and features for inspection, taking into consideration the inheritance hierarchies defined in the metamodel, and the query language furthermore providing a query for relationships between context entities and features for inspection,
a definition of the context subspace by means of the query language being composed of at least one public component and one private component,
the private component defining the underlying connections between the context entities and their relationships, which make up a context subspace, and
the public component marking parts the definition which can be made known to the end user, and describing a predefined situation description,
it being possible to retroactively extend the public component of the definition of the context subspace in the query language such that refinements can be added to the definition by the end user,
refinement of the predefined situation description by the end user by inputting of items of information using the input unit,
termination of the refinement by the end user,
combination of the private components and the public components of the definition of the context subspace, and of the extension resulting from the refinement by the end user, into a query in the query language,
compilation of the query by the computing unit using a context server,
execution of the query by means of the computing unit.

2. A system comprising:
an input unit that is configured for the inputting of items of information by the end user,
a sensor system that is configured to capture at least one item of context information in the surroundings of the end user,
a computing unit that is configured to receive the items of information provided at the input unit and to receive the items of context information captured by means of the sensor system,
the system being designed to execute the method according to claim 1.

3. The system according to claim 2, wherein the sensor system comprises at least one sensor, the sensor being selected from the group comprising: optical sensors, audio sensors, motion sensors, acceleration sensors, locating sensors, magnetic field sensors, orientation sensors, proximity sensors, touch sensors, sensors for temperature, humidity and air pressure, weight sensors, sensors for identifying an activity, gas sensors, odor sensors, biosensors, in particular sensors for capturing vital parameters.

4. The system according to claim 2, characterized in that the input unit comprises at least one touch-sensitive screen and/or one touch-sensitive panel.

5. A computer program product comprising commands which, when the program is executed by the system according to claim 2, cause said system to execute the following method:
provision of an input unit for the inputting of items of information by the end user,
provision of a sensor system, the sensor system being configured to capture at least one item of context information in the surroundings of the end user,
provision of a computing unit, the computing unit being configured to receive the items of information provided at the input unit and to receive the items of context information captured by means of the sensor system, the computing unit furthermore being configured to create a situation description from the items of context information captured by means of the sensor system and to create a context subspace,
capture of at least one item of context information in the surroundings of the end user by means of the sensor system,
creation of a context subspace by means of the computing unit, the context subspace specifying all possible states of context entities and their context relationships to one another, the state of a context entity being described by at least one context attribute, the possible values of the context attribute being specified by a data type, the context relationships being described by at least one context attribute, the possible values of the context attribute being specified by a data type,
context attributes of context entities and context attributes of context relationships being described by means of the items of context information captured by the sensor system,
the context subspace describing the greatest possible extract from a context model that can describe a situation under all possible circumstances,
the context subspace being defined by means of a query language that directly represents an underlying metamodel,
the query language providing a query for context entities of a particular type and features for inspection, taking into consideration the inheritance hierarchies defined in the metamodel, and the query language furthermore providing a query for relationships between context entities and features for inspection,
a definition of the context subspace by means of the query language being composed of at least one public component and one private component,
the private component defining the underlying connections between the context entities and their relationships, which make up a context subspace, and
the public component marking parts the definition which can be made known to the end user, and describing a predefined situation description,
it being possible to retroactively extend the public component of the definition of the context subspace in the query language such that refinements can be added to the definition by the end user,
refinement of the predefined situation description by the end user by inputting of items of information using the input unit,
termination of the refinement by the end user,
combination of the private components and the public components of the definition of the context subspace, and of the extension resulting from the refinement by the end user, into a query in the query language,
compilation of the query by the computing unit using a context server,
execution of the query by means of the computing unit.
